(19) United States
(12) Patent Application Publication
(10) Pub. No.: US 2005/0112651 A1
(43) Pub. Date: May 26, 2005

(54) Microarray comprising a substrate having two-dimensional grating and method of detecting target molecule by using the same

(76) Inventors: Injae Lee, Seongnam-si (KR); Jangseok Ma, Seongnam-si (KR); Soosuk Lee, Suwon-si (KR); Fu Wang, Yongin-si (KR); Youngnam Kwon, Gunpo-si (KR); Huijun Sim, Yongin-si (KR); Younghun Kim, Seoul (KR); Younghwan Lee, Yongin-si (KR)

Correspondence Address: Michael A. Cantor, 55 Griffin South Road, Bloomfield, CT, 06002, US

(21) Appl. No.: 10/968,540

(22) Filed: Oct. 19, 2004

(30) Foreign Application Priority Data
Nov. 20, 2003 (KR) 2003-82642

(51) Int. Cl. C12Q 1/68; C12M 1/34

(52) U.S. Cl. 435/6; 435/287.2; 356/319

(57) ABSTRACT

A microarray including a substrate having a first diffraction grating and a second diffraction grating formed perpendicularly to each other is provided. Also, a method of detecting a target molecule in a sample is provided. The method includes: placing a sample containing a labelled target molecule on a diffraction grating on the above microarray to react with a probe molecule immobilized on the diffraction grating; irradiating a first electromagnetic wave to a product of a reaction between the target molecule and the probe molecule; and detecting a second electromagnetic wave emitted from the labelled probe molecule.

### BACKGROUND OF THE INVENTION

This application claims the benefit of Korean Patent Application No. 2003-82642, filed on Nov. 20, 2003, in the Korean Intellectual Property Office, the disclosure of which is incorporated herein in its entirety by reference.

1. Field of the Invention

The present invention relates to a microarray comprising a substrate having two-dimensional grating formed and a method of detecting a target molecule by using the same.

2. Description of the Related Art

In a microarray, certain molecules are immobilized within discrete known regions on a substrate. Examples of such microarrays include polynucleotide and protein microarrays. In a polynucleotide microarray, a group of polynucleotides is tightly immobilized in a discrete known region on a substrate. Such a microarry is well known in the art, and examples can be found in, for example, U.S. Pat. Nos. 5,445,934 and 5,744,305. Also, it is known that such a microarray is generally manufactured using photolithography. When using photolithography, the polynucleotide microarray can be manufactured by repeatedly exposing an energy source to a discrete known region on a substrate, in which a monomer protected by a removable group is coated, to remove the protecting group, and coupling the deprotected monomer with another monomer protected by the removable group. In this case, the polynucleotide can be immobilized on the polynucleotide microarray by synthesizing a polynucleotide by extending monomers of the polynucleotide one by one or by immobilizing a previously-synthesized polynucleotide in a discrete known region (which is also called a “spotting” method). Such methods of manufacturing a polynucleotide microarray are disclosed in, for example, U.S. Pat. Nos. 5,744,305, 5,143,854, and 5,424,186. This literature regarding polynucleotide microarrays and methods of manufacturing the same is incorporated herein in its entirety by reference.

Conventional integrated-optical chemical and/or biochemical sensors using grating have been developed. For example, U.S. Pat. No. 6,483,096 discloses various sensors having a resonant waveguide structure. According to the patent, a chemical and/or biological substance to be sensed is deposited on a surface of the waveguide structure. Incident light is coupled into the waveguide structure by a grating structure, using a first set of degrees of freedom. The light coupled into the waveguide generates an evanescent wave which exponentially diminishes in the surface of the waveguide and interacts with the substance which is adsorbed into the surface of the waveguide and emits fluorescent light. Fluorescent light is coupled out by the same grating structure, using a second set of degrees of freedom which differs from the first set of degrees of freedom in at least one degree of freedom. By this measure, the emitted outcoupled light is clearly separated from excitation light which is coupled out at a different output angle. However, this conventional technology uses one-dimensional grating and the measured light is coupled out by the waveguide structure and the diffraction grating. Thus, the intensity of the measured light is much weaker than that of fluorescent light which is directly emitted by the excitation light.

The inventors of the present invention found that loss of excitation light can be reduced by using a microarray including a substrate having a two-dimensional grating structure when performing intensive study in order to resolve the above problems in the conventional technologies, and thus completed the present invention.

### SUMMARY OF THE INVENTION

The present invention provides a microarray including a substrate having a two-dimensional grating structure which reduces loss of excitation light.

The present invention also provides a method of detecting a target molecule with a high signal-to-noise ratio by using the above microarray.

According to one aspect of the present invention, there is provided a microarray comprising a substrate having a first diffraction grating and a second diffraction grating formed perpendicularly to each other.

According to another aspect of the present invention, there is provided method of detecting a target molecule in a sample, the method including: placing a sample containing a labelled target molecule on a diffraction grating on a microarray according to an aspect of the present invention to react with a probe molecule immobilized on the diffraction grating; irradiating a first electromagnetic wave onto a product of a reaction between the target molecule and the probe molecule; and detecting a second electromagnetic wave emitted from the labelled probe molecule.

### BRIEF DESCRIPTION OF THE DRAWINGS

The above and other features and advantages of the present invention will become more apparent by describing in detail exemplary embodiments thereof with reference to the attached drawings in which:

FIG. 1 is a magnified top view of a microarray substrate of the present invention;

FIG. 2 is a magnified cross-sectional view of the microarray substrate shown in FIG. 1;

FIG. 3 a plan view of the microarray of FIG. 1;

FIG. 4 is a perspective view of the microarray of FIG. 1

FIG. 5 illustrates various shapes of grating of a microarray substrate according to embodiments of the present invention,

FIG. 6 schematically illustrates that one-dimensional grating prevents localization of light;

FIG. 7 schematically illustrates that localization of light is enhanced by two-dimensional grating; and

FIG. 8 is a graph illustrating that fluorescence intensity is higher for a microarray including a substrate having a two-dimensional grating than for conventional microarrays.

### DETAILED DESCRIPTION OF THE INVENTION

According to an embodiment of the present invention, there is provided a microarray including a substrate on which a first diffraction grating and a second diffraction grating perpendicular to the first diffraction grating are formed and a surface of each of the first and second diffraction gratings is coated with a material having a higher refractive index than the substrate.

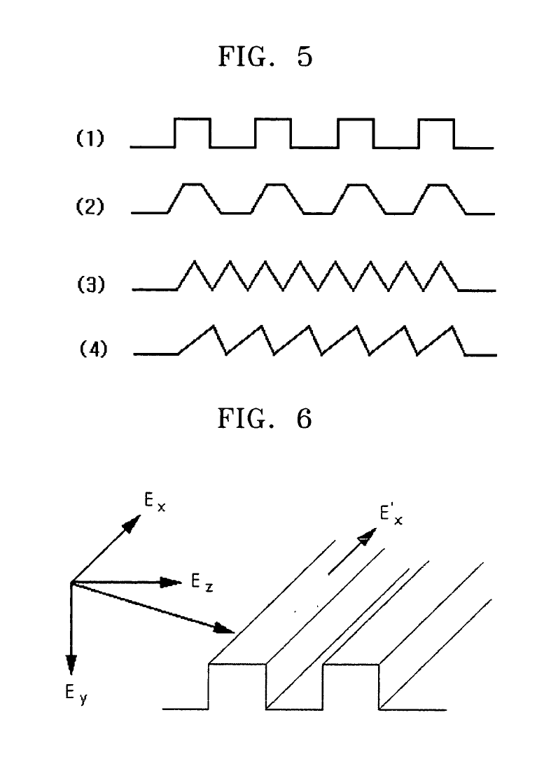

The term “grating” herein refers to a surface having high refractive index, in which a number of grooves (which are also called lines) are etched in parallel. The period of grating may vary according to a desired wavelength range and is generally 600-2000 lines/mm. However, the period of grating is not limited to the above range and may be 300-600 nm. The grating may have a square, trapezoidal, triangular, sine wave, or blaze shape, but is not limited thereto (see FIG. 5). In FIG. 5, (1), (2), (3), and (4) illustrate square-, trapezoidal-, triangular-, and blaze-shaped gratings, respectively.

The high refractive index material may be any material having a higher refractive index than the microarray substrate, and examples of the high refractive index material include TiO₂, Ta₃O₅, HfO₂, ZrO₂, ZnO, and Nb₂O₅. The microarray substrate is conventionally composed of glass, silicone, or plastic materials such as polyethylene, polypropylene, and polystyrene. The microarray includes certain molecules immobilized in a discrete known region on a substrate and may be, for example, a polynucleotide or a protein microarray (see U.S. Pat. No. 5,445,934). For example, the polynucleotide microarray has a substrate in which a group of 10³ or more polynucleotides having different known sequences are covalently bound to a surface in a discrete known region. The group of 10³ or more polynucleotides may occupy 1 cm² or less of the surface of the substrate.

According to another embodiment of the present invention, there is provided a method of detecting a target molecule in a sample, the method including: placing a sample containing a labelled target molecule on a diffraction grating on a microarray according to an embodiment of the present invention to react with a probe molecule immobilized on the diffraction grating; irradiating a first electromagnetic wave onto a product of a reaction between the target molecule and the probe molecule; and detecting a second electromagnetic wave emitted from the labelled probe molecule.

In the method, the microarray may be a polynucleotide or protein microarray. The label may be a light emitting label such as a fluorescent or phosphorescent label.

A probe polynucleotide binding to a specific target polynucleotide sequence is first immobilized on a substrate on which a first diffraction grating and a second diffraction grating are formed perpendicularly to each other and a surface of each gratings is coated with a material having a higher refractive index than the substrate. Thus, a polynucleotide microarray is manufactured. Then, a target molecule in a sample is fluorescence-labelled and the sample is added to the probe polynucleotide, and then hybridisation is performed. After the hybridisation is completed, an unreacted sample is washed and removed. The first electromagnetic wave is irradiated onto the resultant product and the second electromagnetic wave emitted therefrom is measured, thereby detecting the target molecule.

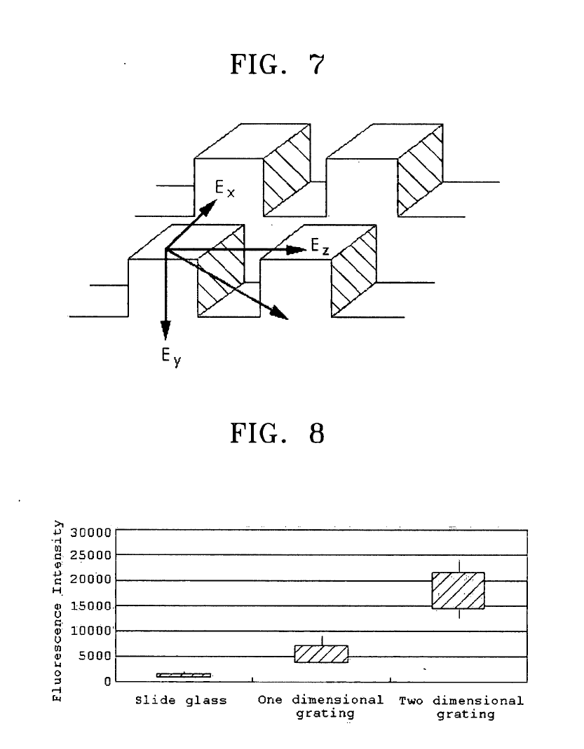

When the first electromagnetic wave is irradiated toward the microarray, the first electromagnetic wave is localized by the two-dimensional grating, thereby reducing loss due to evanescent waves or guided waves. Thus, the light irradiated onto the labelled target molecule is more intense, and therefore a stronger detection signal can be produced than in the conventional technology. The term “localization” herein refers to the trapping of incident light within a region near an incidence location, for example, a region within several microns of an incidence location. FIGS. 6 and 7 schematically illustrate how the localization of light is enhanced by the two-dimensional grating. FIG. 6 schematically illustrates that one-dimensional grating prevents localization of light. Referring to FIG. 6, when incident light is not collinear, a component of the electric field angles in a certain degree against the direction of the grating and some of the light becomes coupled in the direction of grating axis, thereby reducing localization of the first electromagnetic wave, i.e. the excitation light. When the electric field of the incident light has an x₀ component (Ex≠0), this component exits in the direction of the grating axis (x axis) according to boundary condition between the grating and the external atmosphere. FIG. 7 schematically illustrates that localization of light is enhanced by the two-dimensional grating. Some components of the incident electric field under the above condition are trapped in the substrate surface due to the arrangement of the grating structure and form a strong electric field within the substrate, thereby exciting a fluorescent substance adsorbed in the surface of the substrate.

The present invention will now be described in greater detail with reference to the drawings.

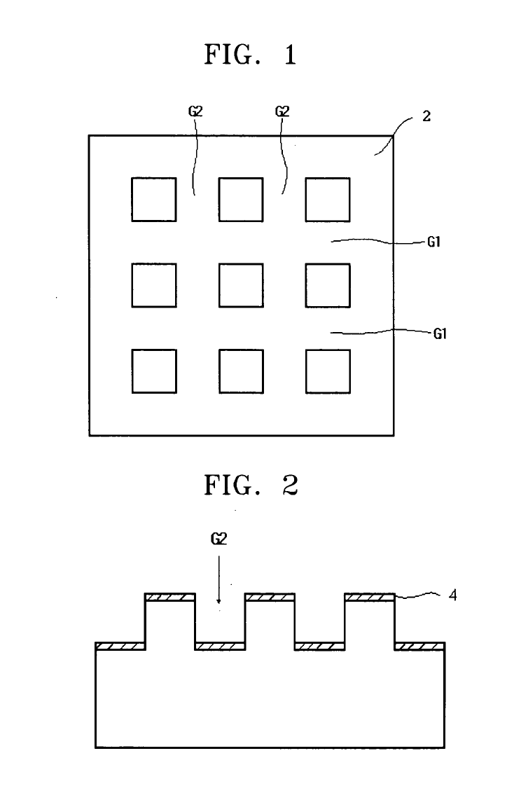

FIG. 1 is a magnified top view of a microarray substrate of the present invention.

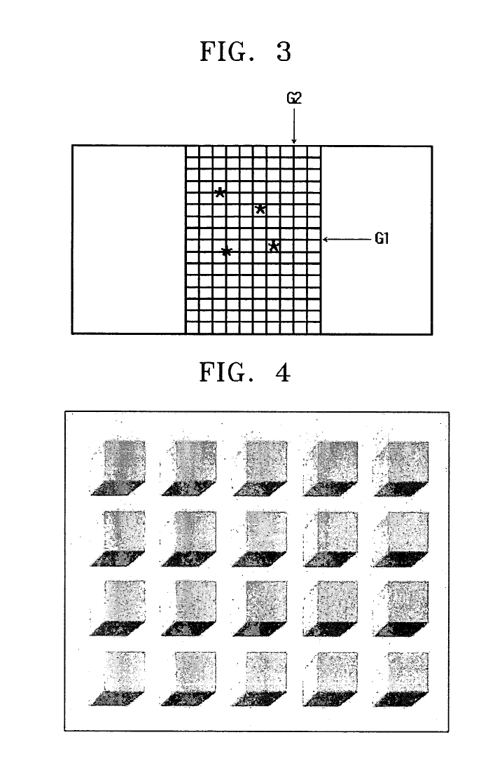

In FIG. 1, first and second gratings G1 and G2 are etched on a substrate 2. FIG. 2 is a magnified cross-sectional view of the microarray substrate shown in FIG. 1. In FIG. 2, the second grating G2 etched into the substrate and a portion of the surface of the substrate that has not been etched are coated with a high refractive index material 4. FIG. 3 is a plan view of the microarray shown in FIG. 1, where grating is represented by lines. In FIG. 3, asterisks indicate where target molecules are bound to probe molecules on the microarray. In the method according to an embodiment of the present invention, the target molecule, for example, may be labelled with a fluorescent label and detected by irradiating the first electromagnetic wave onto the substrate, and then detecting the second electromagnetic wave emitted therefrom. FIG. 4 is a perspective view of the microarray shown in FIG. 1.

The present invention will be described in greater detail with reference to the following example. The following example is for illustrative purposes only, and is not intended to limit the scope of the invention.

### EXAMPLE

In the present Example, 1 μg/ml of BSA which was fluorescence-labelled with Alexa Fluor® 633 (Molecular Probes Inc.) was bound to a substrate on which two-dimensional grating was formed and a high refractive substance was coated. Then, light was irradiated onto the substrate and the emitted fluorescence was measured.

The microarray substrate used in the Example was composed of a glass material. PR patterning was made for one dimensional grating first and by 90 degree rotating the substrate, crossed grating patterning on PR was completed. Two dimensional grating at 500 nm pitch was formed on the glass substrate using a dry etcher. Then, a high refractive index material, TiO₂, was coated to a thickness of 165 nm using an Ion Beam Assisted Coater.

The fluorescence-labelled BSA was immobilized on the substrate with a concentration of 1 μg/ml, light of 633 nm was irradiated onto the microarray, and the emitted fluorescence was detected by a fluorescence reader. As a control, fluorescence was detected in the same manner as described above, except that a glass substrate having no grating and a glass substrate having one-dimensional grating formed were used.

The obtained results are illustrated in FIG. 8. Referring to FIG. 8, the microarray having two-dimensional grating according to an embodiment of the present invention had fluorescence that was about four times greater than the intensity of the fluorescence produced by the microarray having one-dimensional grating. Bars in FIG. 8 represent fluorescence intensity measured for a microarray having a slide glass, a microarray having one-dimensional grating, and a microarray having two dimensional grating, respectively.

The microarray substrate according to an embodiment of the present invention can be used to generate a strong optical signal when using light to perform the detection method with the microarray.

The method of detecting a target molecule using a microarray according to an embodiment of the present invention can be used to obtain a stronger optical signal than on optical signal produced in a conventional optical detection method, thereby efficiently detecting the target molecule.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A microarray comprising a substrate having a first diffraction grating and a second diffraction grating formed perpendicularly to each other.

2. The microarray of claim 1, wherein the grating has a square, trapezoidal, triangular, sine wave, or blaze shape.

3. The microarray of claim 1, wherein a surface of the grating is coated with a high refractive index material having a higher refractive index than the substrate.

4. The microarray of claim 3, wherein the high refractive index material is selected from the group consisting of TiO₂, Ta₃O₅, HfO₂, ZrO₂, ZnO, and Nb₂O₅.

5. The microarray of claim 1, wherein period of the grating is 300-600 nm.

6. The microarray of claim 1, wherein the microarray is a polynucleotide microarry or a protein micro array.

7. A method of detecting a target molecule in a sample, the method comprising:
placing a sample containing a labelled target molecule on a diffraction grating on a microarray of claim 1 to react with a probe molecule immobilized on the diffraction grating;
irradiating a first electromagnetic wave onto a product of a reaction between the target molecule and the probe molecule; and
detecting a second electromagnetic wave emitted from the labelled probe molecule.

8. The method of claim 7, wherein the microarray is a polynucleotide microarray or a protein microarray.

9. The method of claim 7, wherein the label is a fluorescent label.

10. The method of claim 7, wherein the grating has a square, trapezoidal, triangular, sine wave, or blaze shape.

11. The method of claim 7, wherein a surface of the grating is coated with a high refractive index material having a higher refractive index than the substrate.

12. The method of claim 11, wherein the high refractive index material is selected from the group consisting of TiO₂, Ta₃O₅, HfO₂, ZrO₂, ZnO, and Nb₂O₅.

13. The method of claim 7, wherein period of the grating is 300-600 nm.

14. The method of claim 7, wherein the microarray is a polynucleotide microarry or a protein microarray.
